(19) United States
(12) Patent Application Publication
(10) Pub. No.: US 2022/0395378 A1
(43) Pub. Date: Dec. 15, 2022

(54) ANKLE PROSTHESES

(71) Applicant: WRIGHT MEDICAL TECHNOLOGY, INC., Memphis, TN (US)

(72) Inventors: Ramon LUNA, Arlington, TN (US); Shawn E. McGinley, Arlington, TN (US)

(73) Assignee: WRIGHT MEDICAL TECHNOLOGY, INC., Memphis, TN (US)

(21) Appl. No.: 17/774,630

(22) PCT Filed: Oct. 9, 2020
(86) PCT No.: PCT/US2020/054901

§ 371 Date: May 5, 2022

Related U.S. Application Data
(60) Provisional application No. 62/956,699, filed on Jan. 3, 2020.

(51) Int. Cl. A61F 2/42; A61F 2/30

(52) U.S. Cl. CPC A61F 2/4202; A61F 2/30734; A61F 2/30749; A61F 2002/4207; A61F 2002/30245; A61F 2002/3021; A61F 2002/30494; A61F 2002/30332

(57) ABSTRACT

A system includes a first implant component and a second implant component. The first implant component is configured to be secured to a bone and includes a plate and a coupler extending upward from the plate and defining a coupler axis. The second implant component is configured to be coupled to the first implant component. The second implant component includes an articulation surface and defines a cavity configured to receive the coupler of the first implant component. The second implant component is couplable to the first implant component at a plurality of rotational orientations about the coupler axis.

### CROSS-REFERENCE TO RELATED APPLICATION

This application claims priority to U.S. Provisional Patent Application No. 62/956,699, filed on Jan. 3, 2020, the entirety of which is incorporated herein by reference.

### BACKGROUND

An ankle joint may become severely damaged and painful due to arthritis, prior ankle surgery, bone fracture, osteoarthritis, and/or one or more additional conditions. Options for treating the injured ankle have included anti-inflammatory and pain medications, braces, physical therapy, joint arthrodesis, and total ankle replacement.

Total ankle replacement generally comprises two components—a tibial implant and a talar implant. The implants comprise articulation surfaces sized and configured to mimic the range of motion of the ankle joint. For example, the talar implant may comprise an implant sized and configured to mimic the talar dome and the tibial implant may comprise an articulation surface sized and configured to mimic articulation of the tibia.

Installation of a total ankle replacement can include forming one or more holes or cuts in a bone. For example, a hole may be drilled through the talus and into the tibia to create a channel for inserting a tibial stem. In some installations, additional bone is removed from the talus to make space for a talar stem extending from the talar portion.

### SUMMARY

In one aspect, a system includes a first implant component and a second implant component. The first implant component is configured to be secured to a bone and includes a plate and a coupler extending upward from the plate and defining a coupler axis. The second implant component is configured to be coupled to the first implant component. The second implant component includes an articulation surface and defines a cavity configured to receive the coupler of the first implant component. The second implant component is couplable to the first implant component at a plurality of rotational orientations about the coupler axis.

In another aspect, an adaptor for orienting a second implant component to a first implant component includes a top surface, a bottom surface, and an outer surface extending between the top surface and the bottom surface. The adaptor has an indexing feature configured to engage the second implant component to rotationally orient the second implant component.

In another aspect, a method includes attaching a first implant component to a bone. The method further includes selecting a desired rotational orientation for a second implant component relative to the first implant component from a plurality of rotational orientations. The method further includes coupling the second implant component to the first implant component in the desired rotational orientation.

### BRIEF DESCRIPTION OF THE DRAWINGS

These and other features and advantages of the devices and methods described herein will be more fully disclosed in, or rendered obvious by, the following detailed description of the preferred embodiments, which is to be considered together with the accompanying drawings wherein like numbers refer to like parts and further wherein:

FIG. 1 illustrates an anatomic view of an ankle joint.

FIG. 2 illustrates one embodiment of ankle joint having a total ankle replacement system therein.

FIG. 3 is an exploded perspective view of an embodiment of an implant system for a total ankle replacement system.

FIG. 4 is a bottom perspective view of the talar dome of the implant system of FIG. 3.

FIG. 5A is a top view of the implant system of FIG. 3 with the talar dome in a first rotational orientation.

FIG. 5B is a top view of the implant system of FIG. 3 with the talar dome in a second rotational orientation.

FIG. 6 is a cross-sectional exploded view of a talar implant system according to another embodiment in which the coupler of the talar platform is coupled to the plate with a fastener.

FIG. 7 is an exploded view of a talar implant system according to another embodiment.

FIG. 8 is a perspective view of the talar implant system of FIG. 7.

FIG. 9 shows a top view of an implant system, according to another embodiment described herein.

FIG. 10 shows a top view of an implant system, according to another embodiment described herein.

### DETAILED DESCRIPTION

This description of preferred embodiments is intended to be read in connection with the accompanying drawings, which are to be considered part of the entire written description of this invention. The drawing figures are not necessarily to scale and certain features of the invention may be shown exaggerated in scale or in somewhat schematic form in the interest of clarity and conciseness. In the description, relative terms such as “horizontal,” “vertical,” “up,” “down,” “top,” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise.

The present disclosure generally provides a bone implant for use with a joint replacement system. The embodiments described herein include features that allow the articulation surface of the implant to be oriented at a desired rotational orientation from a plurality of potential rotational orientations. Although described herein with reference to an ankle replacement system, the implants described herein can be used in the repair of any joint or bone. Further, a joint replacement system can include two components that allow for rotational alignment or repositioning as described herein. For example, in an ankle replacement system, both the talar implant and the tibial implant can include features that allow the articulation surfaces of the respective implant to be oriented in a desired orientation.

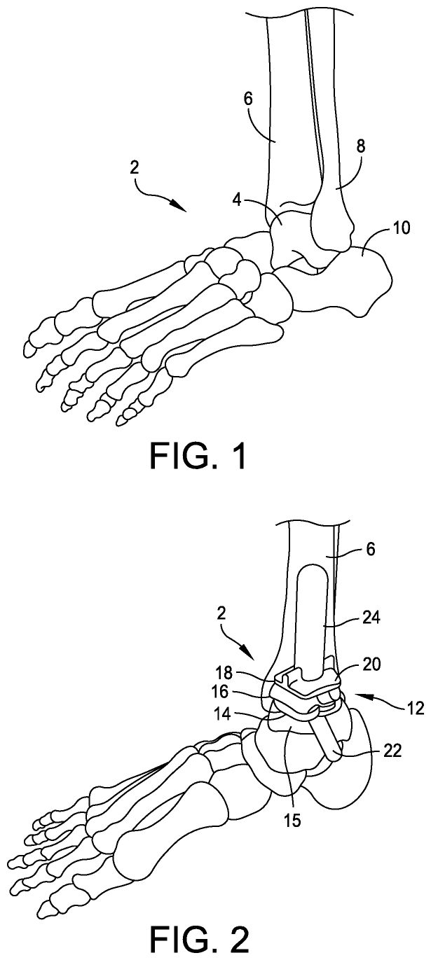

FIG. 1 illustrates an anatomic view of an ankle joint 2. The ankle joint 2 comprises a talus 4 in contact with a tibia 6 and a fibula 8. A calcaneus 10 is located adjacent to the talus 4. In total ankle replacements, the talus 4 and the tibia 6 may be resected, or cut, to allow insertion of a talar implant and a tibial implant.

FIG. 2 illustrates the ankle joint 2 of FIG. 1 having a total ankle replacement system 12 inserted therein. The total ankle replacement system 12 comprises a talar implant 14 and a tibial implant 18. The talar implant 14 comprises a body 15 defining a talar articulation surface 16 (or talar dome). A stem 22 extends into the talus 4 to anchor the talar implant 14 to the talus 4. The tibial implant 18 is sized and configured for installation into the tibia 6. The tibial implant 18 comprises a body having an articulation surface 20 and a tibial stem 24 extending into the tibia 6 to anchor the tibial implant 18. The talar joint surface 16 and the tibial joint surface 20 are mutually sized and configured to articulate. The joint surfaces 16, 20 replace the natural ankle joint surfaces, which are removed, to restore a range of motion and a height that mimics the natural joint. One or more holes may be formed in the tibia and/or the talus prior to and during insertion of the tibial implant 18 or the talar implant 14. For example, in some embodiments, a hole is drilled starting in the bottom of the talus, extending through the talus and into the tibia. The hole may comprise, for example, a 6 mm hole configured to receive the stem 24 of the tibial implant 18.

The joint surfaces 16, 20 may be made of various materials, such as, for example, polyethylene, ultra-high molecular weight polyethylene (UHMWPE), rubber, titanium, titanium alloys, chrome cobalt, surgical steel, and/or any other suitable metal, ceramic, sintered glass, artificial bone, and/or any combination thereof. In some embodiments, the joint surfaces 16, 20 may comprise a coated surface. For example, in some embodiments, the joint surfaces 16, 20 may be plasma sprayed with a closed pore or porous material, The joint surfaces 16, 20 may comprise different materials. For example, the tibial joint surface 20 may comprise a plastic or other non-metallic material and the talar joint surface 16 may comprise a metal surface. Those skilled in the art will recognize that any suitable combination of materials may be used.

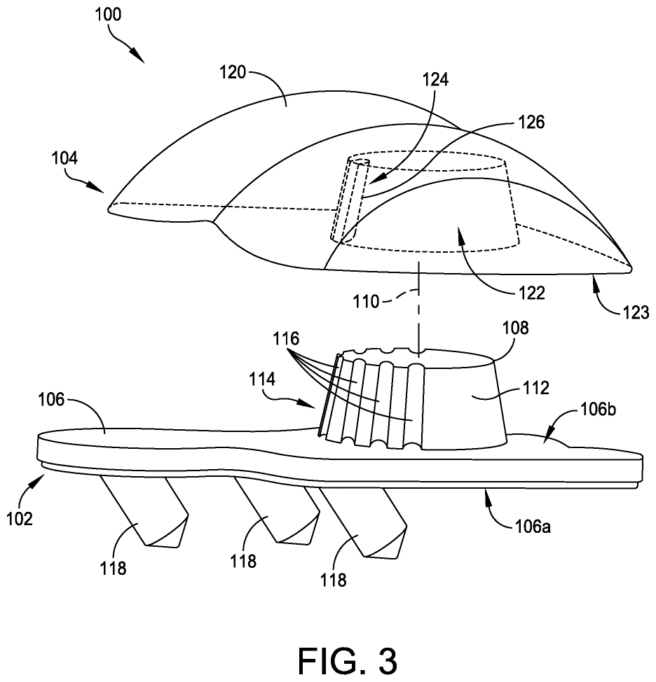

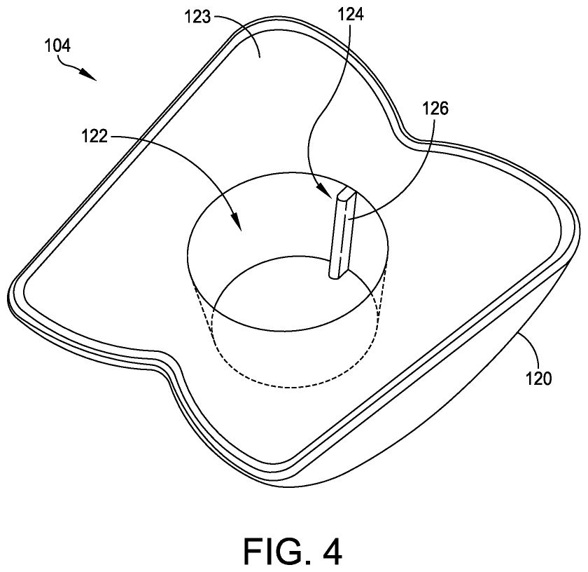

FIG. 3 illustrates one embodiment of a talar implant system 100. The system 100 includes a first implant component, a talar platform 102, and a second implant component, a talar dome 104. The talar platform 102 is configured to be secured to a bone (e.g., a talus). The talar platform 102 can include a plate 106 and a coupler 108 extending upward from the plate 106 and defining a coupler axis 110. The plate 106 can include a first surface 106a configured to be positioned against a bone and a second surface 106b from which the coupler 108 extends. In various embodiments, the coupler 108 can include a tapered or untapered outer surface 112. In various embodiments, the talar platform 102 is a unitary component (i.e., the plate 106 and the coupler 108 are a single component). In other embodiments, described below, the plate 106 and the coupler 108 are separate components and are joined with a fastener.

As shown in FIG. 3, the coupler 108 includes an indexing feature 114. The indexing feature 114 can include, for example, a plurality of notches 116 arranged around all or a portion of the perimeter of the coupler 108. As will be described in further detail herein, the indexing feature 114 allows the talar dome 104 to be coupled to the talar platform 102 at a variety of rotational orientations. This may allow the articulation surface of the talar dome 104 to be positioned in the desired orientation while also maximizing the contact of the plate 106 of the talar platform 102 with the talus, including the talar neck. For example, the talar platform 102 can be positioned on the talus in the optimal position to maximize coverage and contact with the talus. The talar dome 104 can then be coupled to the talar platform 102 in a rotational orientation that provides the optimum contact with the articulation surface of the tibial implant.

The talar platform 102 can further include one or more pegs 118 extending from the plate 106 configured to secure the plate 106 to the talus. The pegs 118 can be angled or straight and can include tapered tips to facilitate insertion into the talus. The talar platform 102 can also include a stem, such as the stem 22 shown in FIG. 2, to couple the talar platform 102 to the talus.

The talar dome 104 is configured to be coupled to the talar platform 102. The talar dome 104 includes an articulation surface 120 configured to contact the articulation surface of the tibial implant (e.g., articulation surface 20) to provide smooth articulation of the ankle joint and to mimic the natural movement of the joint. As shown best in FIG. 4, the talar dome 104 defines a cavity 122 extending into a bottom surface 123. The cavity 122 is configured to receive the coupler 108 of the talar platform 102. The cavity 122 can be tapered to receive a tapered coupler 108 of the talar platform 102. In some embodiments, the coupler 108 and the cavity 122 in the talar dome 104 together form a coupling as in a Morse taper.

The talar dome 104 further includes an indexing feature 124 configured to engage the indexing feature 114 of the talar platform 102. For example, the talar dome 104 can include one or more teeth 126 within the cavity 122, each tooth configured to engage one of the plurality of notches 116 formed in the coupler 108 of the talar platform 102.

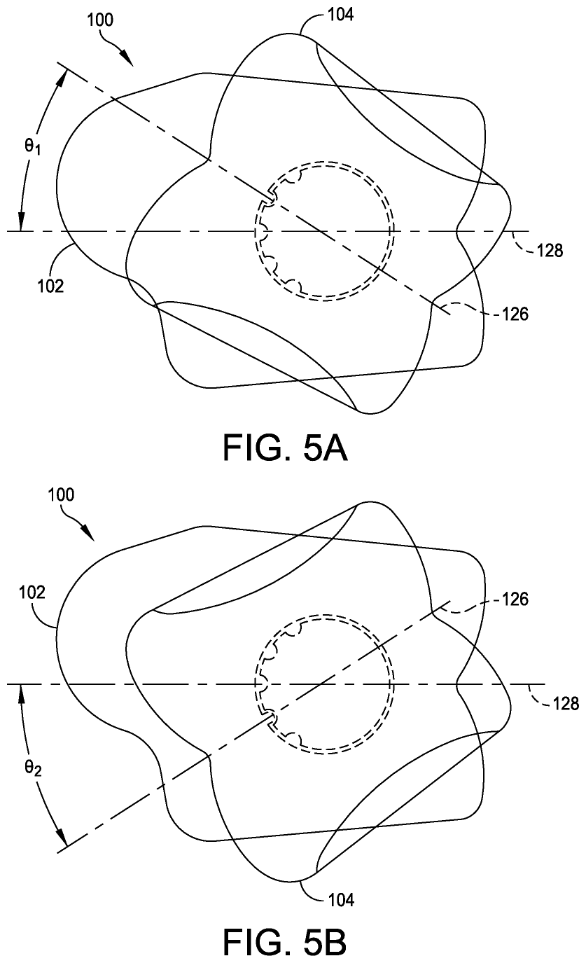

FIGS. 5A and 5B show the talar dome 104 coupled to the talar platform 102 in two exemplary rotational orientations. The system 100 can allow for a range of rotational orientations of the talar dome 104. For example, in various embodiments, the talar dome 104 can be configured such that the centerline 127 of the talar dome 104 forms an angle with the centerline 128 of the talar platform 102 of up to about 25° in each direction. FIG. 5A shows an implant system for the left ankle. In FIG. 5A the talar dome 104 is positioned with the anterior portion of the talar dome 104 rotated medially such that the centerline 127 of the talar dome forms an angle θ₁ with the centerline 128 of the talar platform 102. FIG. 5B shows the talar dome 104 positioned with the anterior portion of the talar dome 104 rotated laterally such that the centerline 127 of the talar dome 104 forms an angle θ₂ with respect to the centerline 128 of the talar platform 102. It should be understood that the orientations and size of the indexing features may be shown in somewhat schematic form in the figures for ease of illustration.

Further, the resolution of the indexing features 114, 124 may allow the talar dome 104 to be oriented at positions that are spaced apart by a desired angle, for example, 1° or 2°, so that the talar dome 104 can be positioned as desired so that the talar dome 104 is positionable in a variety of positions to allow the talar dome 104 to be positioned as desired.

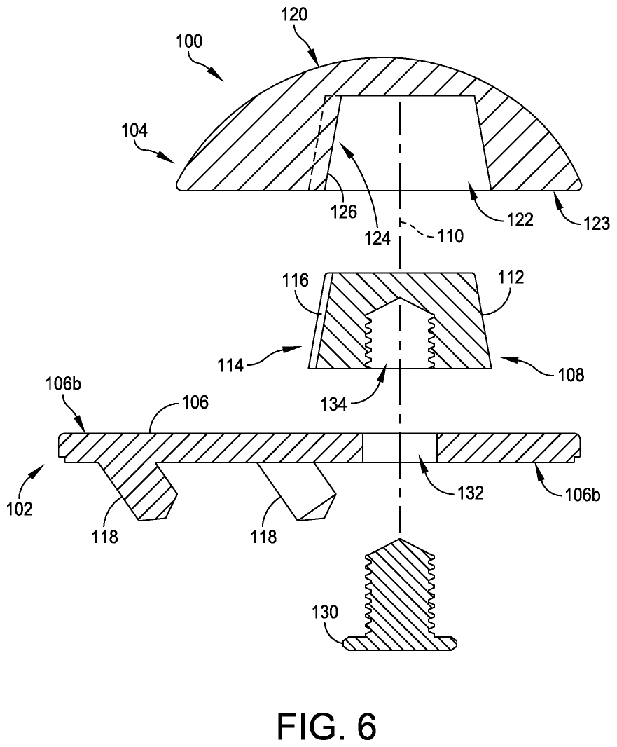

FIG. 6 illustrates an embodiment in which the coupler 108 is coupled to the plate 106 of the talar platform 102 using a fastener 130. The plate 106 defines a fastener aperture 132 extending through the plate 106 configured to allow passage of the fastener 130 and the coupler 108 includes a fastener aperture 134 configured to receive the fastener 130 to couple the coupler 108 to the plate 106. In various embodiments, the fastener 130 is a screw and the fastener aperture 134 in the coupler 108 is threaded to engage the threads of the screw to couple the coupler 108 to the plate 106. In other embodiments, the fastener 130 can be a pin with a barbed shaft configured to engage the interior of the fastener aperture 134 of the coupler 108. The plate 106 and coupler 108 can further include a feature to orient the coupler 108 relative to the plate 106, such as, for example, a pin and pin receiving aperture.

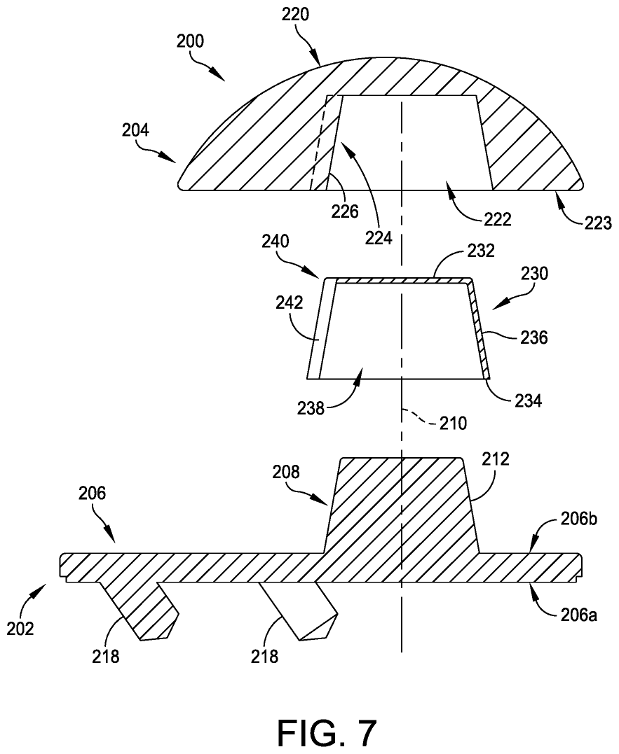

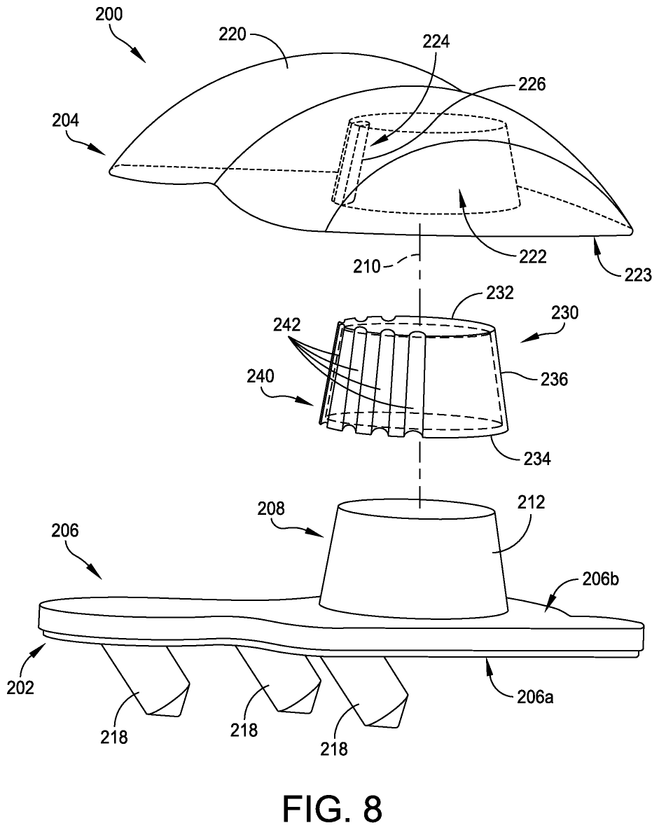

In another embodiment, shown in FIGS. 7 and 8, a talar implant system 200 includes a talar platform 202, a talar dome 204, and an adaptor 230. The talar platform 202 is configured to be secured to a bone (e.g., a talus). The talar platform 202 can include a plate 206 having a first surface 206a configured to be in contact with the bone. The talar platform 202 further includes a coupler 208 extending upward from a second surface 206b of the plate 206 and defining a coupler axis 210. In various embodiments, the coupler 208 can include a tapered or untapered outer surface 212. In various embodiments, the talar platform 202 is a unitary component (i.e., the plate 206 and the coupler 208 are a single component). In other embodiments, as described above, the plate 206 and the coupler 208 are separate components and are joined with a fastener.

The talar platform 202 can further include one or more pegs 218 extending from the plate 206 configured to secure the plate 206 to the talus. The pegs 218 can be angled or straight and can include tapered tips to facilitate insertion into the talus. The talar platform 202 can also include a stem, as shown in FIG. 2, to couple the talar platform 202 to the talus.

The adaptor 230 is configured for orienting the talar dome 204 relative to the talar platform 202. The adaptor 230 includes a top surface 232, a bottom surface 234, and an outer surface 236 extending between the top surface 232 and the bottom surface 234. The adaptor 230 is configured to be received at least partially in the cavity 222 of the talar dome 204. The adaptor 230 defines a recess 238 configured to receive the coupler 208 of the talar platform 202. In various embodiments, the adaptor 230 is frustoconical. In various embodiments, the coupler 208 is tapered and the recess 238 in the adaptor 230 is tapered to receive the coupler 208. The engagement of the coupler 208 and the recess 238 may engage as with a Morse taper to lock the adaptor 230 in position with respect to the talar platform 202.

The adaptor 230 includes an indexing feature 240 configured to orient the talar dome 204 relative to the talar platform 202. The indexing feature 240 can include a plurality of notches 216 formed around all or a portion of the perimeter of the adaptor 230, similar to the indexing feature 114 of the talar platform 102 described above. The indexing feature 240 can include any number of notches 242 with each corresponding to a different rotational orientation of the talar dome 204 relative to the talar platform 202.

The talar dome 204 is configured to be coupled to the talar platform 202 via the adaptor 230. The talar dome 204 includes an articulation surface 220 configured to contact the articulation surface of the tibial implant (e.g., articulation surface 20) to provide smooth articulation of the ankle joint and to mimic the natural movement of the joint. The talar dome 204 defines a cavity 222 configured to receive the adaptor 230. The cavity 222 can be tapered to receive a tapered adaptor 230.

The talar dome 204 further includes an indexing feature 224 configured to engage the indexing feature 240 of the adaptor 230. For example, the talar dome 204 can include a tooth 226 within the cavity 222 configured to engage one of the plurality of notches 242 formed in the adaptor 230.

As described above with respect to the system 100, the system 200 can provide a range of rotational orientations. For example, in various embodiments, the talar dome 204 can be positioned such that the centerline of the talar dome 204 forms an angle of up to about 25° in each direction with respect to the centerline of the talar platform 202. Further, the resolution of the indexing features 224, 240 may allow the talar dome 204 to be oriented at positions that are spaced apart by a desired angle, for example, 1° or 2°.

It should be understood that the indexing features described herein can be provided in other arrangements. For example, the talar dome 104, 204 can have an indexing feature that includes a plurality of notches and the coupler 108 or adaptor 230 can include one or more teeth configured to engage the notches in the talar dome 104, 204 to orient the talar dome 104, 204 with respect to the talar platform 102, 202.

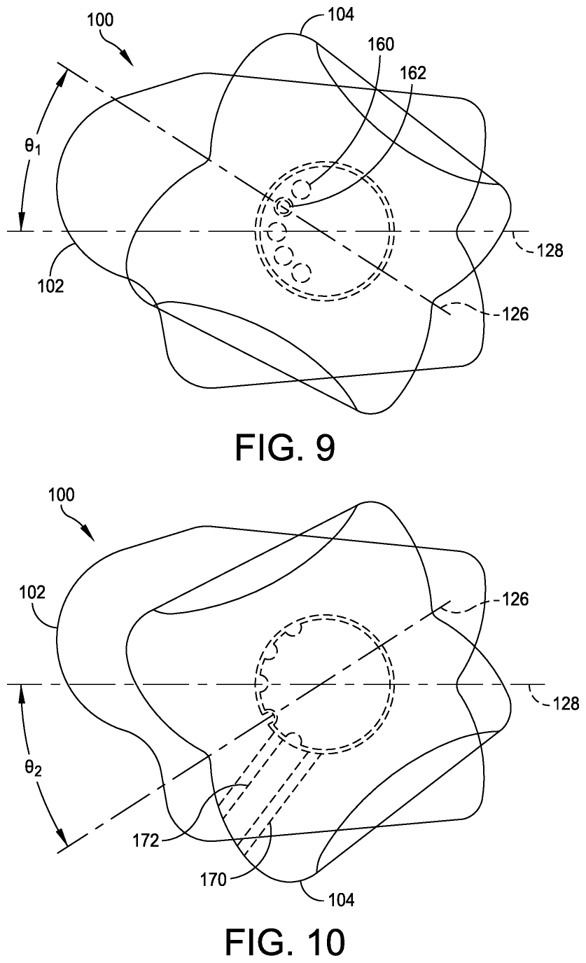

Further, other indexing features can be used in the embodiments described herein. For example, as shown in FIG. 9, in lieu of, or in addition to, the notches 116, the coupler 108 can include a plurality of holes or recesses 160 extending into the coupler 108 from the top face of the coupler 108. The talar dome 104 can include one or more pegs, posts, or pins 162 extending into the cavity 122, with each of the pegs, posts, or pins 162 configured to engage one of the holes or recesses 160 in the coupler 108 to orient the talar dome 104 with respect to the talar platform 102. It should be understood that this can be reversed, with the talar dome 104 including the holes or recesses and the talar platform 102 including the pegs, posts, or pins. Further, the embodiment of FIGS. 7 and 8 can also include indexing features having holes or recesses on either the talar dome 204 or the adaptor 230 and pegs, posts, or pins on the other of the talar dome 204 and the adaptor 230.

In addition, as shown in FIG. 10, the talar dome 104 can include a threaded hole 170 and the implant system 100 can further include a set screw 172 configured to be threadably engaged with the hole 170. With the set screw 172 engaged with the hole 170, rotation of the set screw 172 in a first rotational direction (e.g., clockwise) causes the set screw 172 to engage the coupler 108 to further lock the orientation of the talar dome 104 with respect to the talar platform 102. Rotation of the set screw in the opposite direction (e.g., counter-clockwise) disengages the set screw 172 from the adaptor 108 to allow repositioning of the talar dome 104 with respect to the talar platform 102. In some embodiments, the set screw 172 engages one of the notches 116. Alternatively, the set screw 172 can engage the outer surface 112 of the adapter 108. It should be understood that a set screw such as shown in FIG. 10 can be used with the embodiment shown in FIGS. 7 and 8 as well as the embodiment shown in FIG. 9.

In another aspect, a method of implanting an implant (e.g., a talar implant) includes attaching a first implant component to a bone. The method further includes selecting a desired rotational orientation for a second implant component relative to the first implant component from a plurality of potential rotational orientations. The method further includes coupling the second implant component to the first implant component in the desired rotational orientation.

In various embodiments, the method further includes coupling an adaptor to the first implant component. In such embodiments, the adaptor can include an indexing feature for orienting the second implant component relative to the first implant component. In some embodiments, the indexing feature includes a plurality of notches arranged around a perimeter of the adaptor. In such embodiments, coupling the second implant component to the first implant component includes engaging a tooth of the second implant component with one of the plurality of notches.

In other embodiments, the first implant component includes an indexing feature comprising a plurality of notches arranged around a perimeter of the adaptor. In such embodiments, coupling the second implant component to the first implant component includes engaging a tooth of the second implant component with one of the plurality of notches.

Although the devices, systems, and methods have been described in terms of exemplary embodiments, they are not limited thereto. Rather, the appended claims should be construed broadly, to include other variants and embodiments of the devices, systems, and methods, which may be made by those skilled in the art without departing from the scope and range of equivalents of the devices, kits, systems, and methods.

What is claimed is:

1. A system, comprising:
a first implant component configured to be secured to a bone, the first implant component comprising a plate and a coupler extending upward from the plate and defining a coupler axis; and
a second implant component configured to be coupled to the first implant component, the second implant component comprising an articulation surface and defining a cavity configured to receive the coupler of the first implant component;
wherein the second implant component is couplable to the first implant component at a plurality of rotational orientations about the coupler axis.

2. The system of claim 1, wherein the first implant component is a talar platform and is configured to be secured to a talus bone.

3. The system of claim 1, wherein the second implant component is a talar dome.

4. The system of claim 1, wherein the cavity includes a tapered inner surface, and wherein an outer surface of the coupler is tapered to engage the inner surface of the cavity.

5. The system of claim 1, wherein the coupler and the plate of the first implant component are joined together using a fastener.

6. The system of claim 1, further comprising an adaptor for orienting the second implant component relative to the first implant component, the adaptor comprising a top surface, a bottom surface, and an outer surface extending between the top surface and the bottom surface, wherein the adaptor is configured to be received at least partially in the cavity of the second implant component, and wherein the adaptor defines a recess configured to receive the coupler of the first implant component.

7. The system of claim 6, wherein the adaptor is frustoconical.

8. The system of claim 6, wherein the coupler is tapered and the recess in the adaptor is tapered to receive the coupler.

9. The system of claim 6, wherein the second implant component includes a second implant indexing feature configured to engage an adaptor indexing feature of the adaptor to orient the second implant component relative to the first implant component.

10. The system of claim 9, wherein the second implant indexing feature comprises a tooth and the adaptor indexing feature comprises a plurality of notches arranged around a perimeter of the adaptor, and wherein the tooth is configured to engage one of the plurality of notches to orient the second implant component relative to the first implant component.

11. The system of claim 1, wherein the second implant component includes a second implant indexing feature configured to engage a first implant indexing feature of the coupler to orient the second implant component relative to the first implant component.

12. The system of claim 11, wherein the second implant indexing feature comprises a tooth and the first implant indexing feature comprises a plurality of notches arranged around a perimeter of the coupler, and the tooth is configured to engage one of the plurality of notches to orient the second implant component relative to the first implant component.

13. The system of claim 1, wherein the plurality of rotational orientations includes a first rotational orientation and a second rotational orientation and wherein the first and second rotational orientations differ by a rotation of up to 50° about the coupler axis.

14. An adaptor for orienting a second implant component relative to a first implant component, the adaptor comprising a top surface, a bottom surface, and an outer surface extending between the top surface and the bottom surface, the adaptor having an indexing feature configured to engage the second implant component coupled to rotationally orient the second implant component.

15. The adaptor of claim 14, wherein the indexing feature comprises a plurality of notches arranged around a perimeter of the adaptor, and wherein each of the plurality of notches are configured to receive a tooth of the second implant component to orient the second implant component relative to the adaptor.

16. The adaptor of claim 14, wherein the adaptor defines a recess configured to receive a coupler of a talar platform.

17. A method, comprising:
attaching a first implant component to a bone;
selecting a desired rotational orientation for a second implant component relative to the first implant component from a plurality of potential rotational orientations; and
coupling the second implant component to the first implant component in the desired rotational orientation.

18. The method of claim 17, further comprising coupling an adaptor to the first implant component, wherein the adaptor includes an indexing feature for orienting the second implant component relative to the first implant component.

19. The method of claim 18, wherein the indexing feature comprises a plurality of notches arranged around a perimeter of the adaptor, and wherein coupling the second implant component to the first implant component includes engaging a tooth of the second implant component with one of the plurality of notches.

20. The method of claim 17, wherein the first implant component includes an indexing feature comprising a plurality of notches arranged around a perimeter of the first implant component, and wherein coupling the second implant component to the first implant component includes engaging a tooth of the second implant component with one of the plurality of notches.
